(19) United States
(12) Patent Application Publication
(10) Pub. No.: US 2010/0311830 A1
(43) Pub. Date: Dec. 9, 2010

(54) Menthol Replacement

(75) Inventors: Marco DE JONGE, Utrecht (NL); Cornelis Winkel, Bussum (NL); Sarah Barrie Machover, Cincinnati, OH (US); Stefan Michael Furrer, Cincinnati, OH (US); Alexander P. Tondeur, Loosdrecht (NL)

Correspondence Address: CURATOLO SIDOTI CO., LPA, 24500 CENTER RIDGE ROAD, SUITE 280, CLEVELAND, OH, 44145, US

(73) Assignee: Givaudan S.A., Vernier (CH)

(21) Appl. No.: 12/783,607

(22) Filed: May 20, 2010

Related U.S. Application Data
(60) Provisional application No. 61/179,919, filed on May 20, 2009.

(51) Int. Cl. A61K 31/215; A61K 31/164; A61K 31/165; C07C 35/12; A61P 43/00

(52) U.S. Cl. 514/546; 514/629; 514/622; 568/829

(57) ABSTRACT

A method of producing in a composition adapted to be applied to the mucous membranes, the physiological effects of menthol at a reduced menthol concentration, by the partial replacement of menthol by a cooling and flavour blend, including
(a) at least one compound selected from the group consisting of lactoyl ethanolamide, nonanoic acid vanillyl amide and menthyl lactate; and
(b) at least one compound of the Formula I

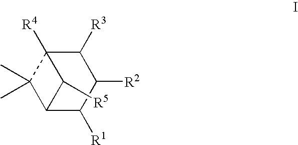

in which the dotted line represents an optional single bond, the moieties R¹, R², R³, R⁴ and R⁵ being independently selected from H, CH₃ and OH, three moieties being H, one CH₃ and one OH, such that
(i) when the optional single bond is absent, one of R¹ and R⁵ is OH only when R³ is H; and
(ii) when the optional single bond is present, R³ is CH₃ and one of R¹ and R⁵ is OH.

This application claims the benefit under 35 U.S.C. 119(e) of U.S. Provisional Application for Patent Ser. No. 61/179,919, filed May 20, 2009, incorporated herein by reference.

This disclosure relates to a method for replacing menthol.

Menthol, 2-isopropyl-5-methylcyclohexanol, is a compound that occurs naturally and can also be made synthetically. Menthol has eight stereoisomers, all of which have some degree of cooling properties. The compound most commonly used is l-menthol (also known as laevo-menthol and (−)-menthol), and all references to menthol in this disclosure are references to l-menthol.

Menthol is well known for its cooling effect on the skin and the mucous membranes of the mouth. As a result, it has been widely used in many compositions to be applied to the skin, or to be taken orally or ingested, for example, confectionery, baked goods, mouthwashes, dentifrices, medical and cosmetic creams and skin lotions. It also adds a distinct and desirable odour and flavour.

While menthol has proved very useful in these applications, it can prove disadvantageous in some oral applications, where, when used in high amounts, it can give a bitter off-note. In addition, it can be quite expensive.

Other materials have been suggested as partial menthol replacements. Examples include lactoyl ethanol amide and nonanoic acid vanillyl amide, these having the advantage of being cheaper than menthol. However, they are not as effective and they are generally used as auxiliary materials, lactoyl ethanol amide enhancing cooling power in the initial phase and nonanoic acid vanillyl amide enhancing the cooling aftertaste. Known cooling compounds, such as menthyl lactate and WS-3™, can also be used, and are highly effective, but these are expensive materials relative to menthol, and this tends to confine them to specialised applications where their performance can justify the higher cost. In addition, with an increased desire for natural materials, the presence of synthetic compounds such as WS-3 is not so desirable.

It has now been found that the desired effect of menthol as both coolant and flavour can be achieved at a reduced menthol concentration. There is therefore provided a method of producing in a composition adapted to be applied to the mucous membranes, the physiological effects of menthol at a reduced menthol concentration, comprising the partial replacement of menthol by a cooling and flavour blend, comprising
(a) at least one compound selected from the group consisting of lactoyl ethanolamide, nonanoic acid vanillyl amide and menthyl lactate; and
(b) at least one compound of the Formula I

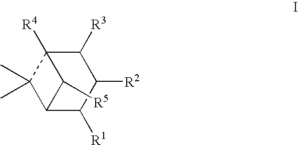

in which the dotted line represents an optional single bond, the moieties R¹, R², R³, R⁴ and R⁵ being independently selected from H, CH₃ and OH, three moieties being H, one CH₃ and one OH, such that
(i) when the optional single bond is absent, one of R¹ and R⁵ is OH only when R³ is H; and
(ii) when the optional single bond is present, R³ is CH₃ and one of R¹ and R⁵ is OH.

By “partial replacement” is meant that, while a considerable proportion of menthol may be replaced, complete replacement may not be possible. Typically, from about 25% to about 75% by weight of menthol may be replaced. Replacement of less than 25% is possible, but there is no economic advantage to the replacement. The determination of the appropriate replacement proportion may be determined in every case by routine, non-inventive experimentation. However, as a general rule, up to 50% replacement is possible in all cases.

Lactoyl ethanolamide, nonanoic acid vanillyl amide and menthyl lactate are all well known in the art as compounds that have an effect on the skin and the mucous membranes, and have been widely used in such applications.

The compounds of Formula I do not have significant cooling properties (some have none), but all provide a menthol flavour. They are also relatively volatile and contribute a menthol-like odour, so that the menthol perception is not significantly reduced.

A table of typical compounds of Formula I (when the optional bond is absent) is shown in Table 1 below:

TABLE 1

| Compound | R1 | R2 | R3 | R4 | R5 |
|---|---|---|---|---|---|
| A | H | OH | Me | H | H |
| B | OH | Me | H | H | H |
| C | OH | H | H | Me | H |
| D | OH | H | H | H | Me |
| E | Me | OH | H | H | H |
| F | H | OH | H | Me | H |
| G | H | OH | H | H | Me |
| H | H | Me | OH | H | H |
| I | Me | H | OH | H | H |

Compounds C and G are novel compounds. There is therefore also provided a compound of the Formula I as hereinabove defined, in which R² is OH, R³ and R⁴ are H and R¹ and R⁵ are selected such that one is H and the other CH₃.

Compounds A-D, F, H and I are known compounds, none of which are themselves known to have menthol-like effects. All are either available or may be readily prepared by known synthetic methods. Typical examples of preparations of some of these compounds are as follows:

### Compound A

5-Isopropyl-2-methylcyclohexanol can be conveniently prepared by known methods, for example, Kergomard, FR 1183850, 1959 or Hueckel, et al., Justus Liebig's Annalen Der Chemie, 1965, 687, 85-102.

### Compound E

2-Methyl-3-Isopropylcyclohexanol can be prepared by known hydrogenation methods of 2-Methyl-3-Isopropylphenol, for example, M. Carpenter, et al., Journal of Organic Chemistry, 1955, 20, 401-411 or R. Lamartine, et al., Bulletin de la Societe Chimique de France, 1969, 2, 437-442.

### Compound G

3-Isopropyl-4-Methylcyclohexanol can be prepared by known hydrogenation methods of 3-Isopropyl-4-Methylphenol, for example, R. Lamartine, FR 1523471, 1968 or M Newman, et al., Journal of Organic Chemistry, 1963, 28(10), 2804-2806.

A particular example of such a compound is Compound A, known under the trivial name carvomenthol. The compound comprising the optional bond is known as verbanol. Verbanol may be prepared by a number of known methods, such as the reduction of verbenone with sodium in moist ether solution (Blumann and Zeitschel, Ber, 1913, 46, 1192).

The compounds hereinabove described all possess at least one chiral centre, and are therefore available in at least one enantiomeric form (in the case of verbanol and carvomenthol, there are respectively 16 and 8 possible isomers). For example, a typical synthesised carvomenthol had the following proportions (measured by GC/NMR):

| | | |
|---|---|---|
| I | 63.69% | 1R,2R,4R & 1S,2S,4S Carvomenthol |
| II | 14.63% | 1S,2S,4R & 1R,2R,4S iso-Carvomenthol |
| III | 11.54% | 1S,2R,4R & 1R,2S,4S neo-iso-Carvomenthol |
| IV | 8.69% | 1R,2S,4R & 1S,2R,4S neo-Carvomenthol. |

For many uses, a racemic mixture is suitable; this has the additional advantage of reduced costs, because it is not necessary to separate the individual isomers. However, if desired, resolution of the enantiomers may be achieved by art-recognised methods. This adds to the expense of the compounds, but it may be justified in some individual cases.

The method hereinabove described may be used in respect of products that are applied to the mucous membranes such as oral mucosa. By “applying” is meant any form of bringing into contact, for example, oral ingestion, or, in the case of tobacco products, inhalation. There is therefore also provided a method of providing a cooling sensation to mucous membranes by applying thereto a product comprising an effective amount of a cooling composition as hereinabove described.

Products that are applied to the oral mucosa include, but are not limited to foodstuffs and beverages taken into the mouth and swallowed, and products taken for reasons other than their nutritional value, e.g. tablets, lozenges, mouthwashes, throat sprays, dentifrices and chewing gums, which may be applied to the oral mucosa for the purpose of cleaning, freshening, healing, and/or deodorising.

Particular examples of foodstuffs and beverages include, but are not limited to frozen confectionery such as ice creams and sorbets; desserts such as jelly and pudding; confectionery such as cakes, cookies, chocolates, and chewing gum; jams; candies; breads; tea beverages such as green tea, black tea, chamomile tea, mulberry leaf tea, rooibos tea, peppermint tea; soups; seasonings; instant beverages; snack foods and the like.

Further examples of products that are applied to the oral mucosa may include, but are not limited to, oral care products such as toothpastes, tooth gels, tooth powders, tooth whitening products, dental floss, anti-plaque and anti-gingivitis compositions, compositions for treatment of nasal symptoms, and the like.

The overall quantities of the compounds (a) and (b) that may be present in a product, and the relative proportions of (a) and (b) that may be used will depend on the nature of that product and by the nature and intensity of the effect desired. They may vary over wide limits, and the skilled person may determine the quantities and appropriate relative proportions in every single case, with only routine experimentation. However, as a rough guide, the relative weight proportion may vary between about 5:1 and about 1:100. The following proportions are given as a general guideline for particular applications:

| | | Quantity of | Quantity of |
|---|---|---|---|
| | | (a) + (b) | (a) + (b) + menthol |
| Product | Ratio (a):(b) | in product (ppm) | in product (ppm) |
| chewing gum | 1.5:1-30:1 | 5-75 | 35-200 |
| boiled sweets | 5:1-100:1 | 10-100 | 100-750 |
| toothpaste | 5:1-100:1 | 10-100 | 100-750 |
| mouthwash | 5:1-100:1 | 10-100 | 100-750 |

In addition, as previously mentioned, the proportion of menthol replaced may be varied widely, typically from about 25% to about 75% by weight, depending again on the desired effect. That is, the combination of lactoyl ethanolamide, nonanoic acid vanillyl amide and menthyl lactate, and the compound of Formula I may comprise from about 25% to about 75% by weight of the total weight of menthol, lactoyl ethanolamide, nonanoic acid vanillyl amide, menthyl lactate and the compound of Formula I. As a general, non-limiting guide, usage of the compound (in addition to menthol) in most consumer products will vary between about 0.01 and about 100 ppm. In particular examples, the quantities may vary between about 0.05 and about 75 ppm, and in other examples, between about 0.1 and about 50 ppm.

Such commercial products may include all the art-recognised ingredients usual in such products, in art-recognised proportions.

The disclosure is now further described with reference to the following non-limiting examples, in which all parts are expressed by weight.

### EXAMPLE 1

A chewing gum coating is made according to the following formulation:

### Basic Recipe:

| | | |
|---|---|---|
| Mix 1 | Icing Sugar | 235 parts. |
| | Dextrose | 235 parts. |
| Mix 2 | Gelatine 230 Bloom 15% soln | 30 parts. |
| | Flavour | 1 part. |

### Processing:

All of the powders were put in a Z-blade mixer at a mixer temperature heated to 50° C. and mixed. During this mixing, the liquid components were added. Kneading was continued for about 3 minutes, and the resulting paste was rolled to the desired thickness. It was then sliced into comprimates and allowed to dry for 5 hours at 45° C.

To samples of this formulation were added 1 part of two flavouring compositions, one of which was a standard mint flavouring with menthol and the other of which had 50% of the menthol replaced. The formulations were as follows:

### Standard Flavouring

| | |
|---|---|
| peppermint oil yakima | 700 parts |
| l-menthol | 300 parts |

### 50% Replacement Flavouring

| | | |
|---|---|---|
| peppermint oil yakima | 700 | parts |
| menthol | 150 | parts |
| lactoyl ethanolamide | 30 | parts |
| nonanoic vanillyl amide | 0.04 | parts |
| carvomenthol | 1 | part |
| triacetin | 118.96 | parts. |

Dosage of the flavouring was 0.2% by weight of the chewing gum coating.

The two chewing gum coating formulations were tested by a sensory panel consisting of 50 people, comprising 25 experts (flavourists, application specialists) and 25 consumers. The panel was unable to distinguish between the 2 samples.

### EXAMPLE 2

Example 1 was repeated, using the following menthol and menthol replacement flavours:

### Menthol Replacement Formula:

| | | |
|---|---|---|
| menthol | 500 | parts |
| lactoyl ethanolamide | 100 | parts |
| nonanoic acid vanillyl amide | 0.2 | parts |
| carvomenthol | 5 | parts |
| triacetin | to 1000 | parts |

### Peppermint Menthol Replacement Flavour

| | | |
|---|---|---|
| peppermint oil yakima | 700 | parts |
| menthol | 150 | parts |
| lactoyl ethanolamide | 30 | parts |
| nonanoic vanillyl amide | 0.04 | parts |
| carvomenthol | 1 | part. |

The replacement rate was such that 1000 parts of the menthol formula was replaced by 605.2 parts of the replacement formula, plus 394.8 parts triacetin.

The testing panel of Example 1 found no discernible difference between the two formulae.

### EXAMPLE 3

Example 2 was repeated, but with the substitution of the carvomenthol by an equal quantity of Verbanol.

A testing panel of 44 people was used to test the compositions. Of these, 17 were able to discern a difference, the other 27 were not.

Although the embodiments have been described in detail through the above description and the preceding examples, these examples are for the purpose of illustration only and it is understood that variations and modifications can be made by one skilled in the art without departing from the spirit and the scope of the disclosure. It should be understood that the embodiments described above are not only in the alternative, but can be combined.

1. A method of producing in a composition adapted to be applied to the mucous membranes, the physiological effects of menthol at a reduced menthol concentration, comprising the partial replacement of menthol by a cooling and flavour blend, comprising:
(a) at least one compound selected from the group consisting of lactoyl ethanolamide, nonanoic acid vanillyl amide and menthyl lactate; and
(b) at least one compound of the Formula I 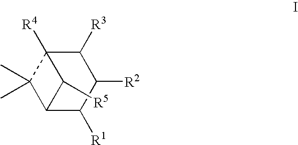
in which the dotted line represents an optional single bond, the moieties R¹, R², R³, R⁴ and R⁵ being independently selected from H, CH₃ and OH, three moieties being H, one CH₃ and one OH, such that
(i) when the optional single bond is absent, one of R¹ and R⁵ is OH only when R³ is H; and
(ii) when the optional single bond is present, R³ is CH₃ and one of R¹ and R⁵ is OH.

2. A compound of the Formula I 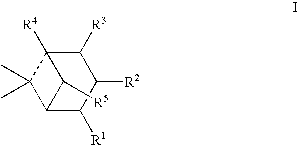
in which R² is OH, R³ and R⁴ are H and R¹ and R⁵ are selected such that one is H and the other is CH₃.

3. A consumer product of the type adapted to provide a cooling effect to the skin or the mucous membranes of the mouth and nose, comprising
(a) menthol;
(b) at least one compound selected from the group consisting of lactoyl ethanolamide, nonanoic acid vanillyl amide and menthyl lactate; and
(c) at least one compound of the Formula I 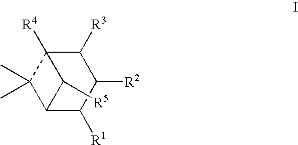
in which the dotted line represents an optional single bond, the moieties R¹, R², R³, R⁴ and R⁵ being independently selected from H, CH₃ and OH, three moieties being H, one CH₃ and one OH, such that
(i) when the optional single bond is absent, one of R¹ and R⁵ is OH only when R³ is H; and
(ii) when the optional single bond is present, R³ is CH₃ and one of R¹ and R⁵ is OH.

4. The product according to claim 3, in which the combination of lactoyl ethanolamide, nonanoic acid vanillyl amide and menthyl lactate, and the compound of Formula I comprises from about 25% to about 75% by weight of the total weight of menthol, lactoyl ethanolamide, nonanoic acid vanillyl amide, menthyl lactate and the compound of Formula I.

5. The product according to claim 3, in which the quantity of lactoyl ethanolamide, nonanoic acid vanillyl amide, menthyl lactate, and the compound of Formula I in the product is from about 0.01 to about 100 ppm.
